SEQUENCE LISTING (12 sequences)

SEQ ID NO: 1 - protein, 451 aa, Streptomyces albidoflavus
VGAEGAERDAVGALFEELVREHRVTGAQLSVYRDGALSEYATGLASVRTGEPVTPRTGFPFGSVTKFLTAELVMQFVCDGDLDLDDPLAGLLPDLGRAAGPPLGTATVRQLLSHTAGVVDSIEYDEMRGPSYRRFAAACARQPALFPPGLAFSYSNTGYCLLGAVIEAASGMDWWTAMDSCLLRPLGIEPAFLHDPRPGQGGAARPVAEGHALRAGGERAEHVDHMASLSLAAAGGLVGSATDLVTAARPHLADRKTFAQHDLLPEDAVLAMRTCVPDAEPFGLADGWGLGLMRHGTGDGAWYGHDGAVGGASCNLRIHPDRSLALALTANSTAGPKLWEALVARLPEAGLDVGHYALPVPDSAPLAPDAGHLGTYANGDLELMVTHDAAGDLFLTRESYSDYRLSLHEDDLFVARSGEPGALPITGRFVREHPAGPVALLQYGGRAMHRL

SEQ ID NO: 2 - DNA, 1356 bp, Streptomyces albidoflavus
gtgggtgccgagggggcggaacgggacgcggtcggcgcgctcttcgaggagctggtgcgcgagcaccgggtcaccggggcgcagctgtccgtctaccgcgacggcgccctcagcgagtacgccaccggcctcgcctcggtccgcaccggtgaaccggtcaccccccggacgggtttccccttcggctcggtgaccaagttcctcaccgccgagctggtcatgcagttcgtctgcgacggcgacctggacctcgacgatcccctcgccgggctcctccccgacctggggcgcgccgccggcccgcccctcggcaccgccaccgtccgccagctcctcagccacaccgccggggtggtggacagcatcgagtacgacgagatgcgcggtccctcctaccggcggttcgccgcggcgtgcgcccggcagcccgcgctcttcccgccgggcctcgccttctcctactccaacaccgggtactgcctgctgggcgcggtgatcgaggcggcgtcggggatggactggtggacggcgatggacagttgcctgctgcgtccgctcggcatcgagccggccttcctgcacgacccgcgccccggccagggcggcgccgcccggccggtggccgagggccacgcgctgcgcgccggcggcgagcgggccgagcacgtcgaccacatggcctcgctctcgctggccgccgccggcgggctggtcggcagcgccaccgacctggtcaccgcggcccgcccgcacctggccgaccggaagaccttcgcccagcacgacctgctccccgaggacgccgtcctcgccatgcgcacctgcgtgccggacgccgagccgttcggcctggccgacggctgggggctgggcctgatgcgccacggcaccggcgacggcgcctggtacggccacgacggcgcggtgggcggcgcctcctgcaacctccgtatccacccggaccgttcgctggcgctggcgctgaccgccaactccaccgccgggccgaagctgtgggaggcgctggtcgcgcggctgccggaggccgggctcgacgtgggccactacgcgctgcccgtccccgactccgcgccgctggccccggacgccggccacctcggcacctacgccaacggcgacctggagctgatggtgacccacgacgccgccggcgacctcttcctgacccgcgagagctactccgactaccgcctctccctgcacgaggacgacctcttcgtggcccggagcggcgagcccggcgcgctcccgatcaccggccgcttcgtccgggagcacccggccgggccggtcgccctgctccagtacggcggccgggccatgcaccggctctga

SEQ ID NO: 3 - DNA, 30 bp, Artificial Sequence
  Forward primer
ccggaattccatatgggtgccgagggggcg

SEQ ID NO: 4 - DNA, 27 bp, Artificial Sequence
  Reverse primer
cccaagctttcagagccggtgcatggc

SEQ ID NO: 5 - protein, 432 aa, Goodfellowiella coeruleoviolacea
VPNEQDLGSLLARLAERHRVPGAQLSVCHDSASVTVVTGRERHGAATPVTARSAFPLGSLTKPFTAALAMVLVADGDLDLDEPVAGYHPGVTLRRLLSHTAGLESNVDEQAAAGVPRRRWVTRYPARLAPEHPPGTVFSYSNVGYLLAGHLVEEVTGLDWAEAVESVLLRPLGITPAFVVGAAGSRGVVCGHVLTDDRVLPVAEQVMPEVEAPNGALALSSADLLAFAALFSANSPAPDVLDPATAALMCQDQLADVRVGPYGMADGWGLGWARYRGAGADWFGHDGTGDGTSCHLRFDPERGTAVALTTNGSTGAALWRELCAELAERHVLPPEWGREPGEATPVAGPPDGAGHYANGPAEFVVTRQDDDGLALVLDGAQAVPLTCLPDLRFQLRLGTTVLSGRFLRDPDTGGIDHIQVTGRLARRRQVTG

SEQ ID NO: 6 - DNA, 1299 bp, Goodfellowiella coeruleoviolacea
gtgcccaacgagcaggatctcgggtcgctgctggcccggctggccgaacgccaccgggtccccggcgcccagctcagcgtctgccacgacagcgcctcggtcacggtggtgaccggccgggaacgccacggcgcggccacgccggtcaccgcgcggtcggcgttcccgctggggtcgctcaccaagccgttcaccgcggcgctggccatggtcctggtcgccgacggcgatctggacctcgacgagccggtggccggctatcaccccggggtgaccctgcgccgcctgctcagtcacaccgccgggctggagtccaacgtggacgaacaggccgcggccggggtgccgcgccgccgctgggtgacccgctacccggcgcggctggccccggagcacccgccgggcacggtgttctcctactccaacgtcggttacctgctggccggccacctggtggaggaggtgaccggactggactgggccgaggcggtggagtcggtgctgctgcgaccgctgggcatcaccccggcgttcgtggtgggcgccgcgggcagccgtggagtggtgtgcgggcacgtgctgaccgacgaccgggtgctgcccgtggccgagcaggtgatgcccgaggtcgaggcgcccaacggcgcgctcgcgctgtcctcggccgacctgctcgccttcgcggcgctgttctcggcgaactccccggcccccgacgtgctcgacccggccacggccgcgctgatgtgccaggaccagctcgccgacgtgcgggtcggcccgtacggcatggccgacgggtggggcctgggctgggcgcgctaccgcggcgcgggcgccgactggttcggccacgacggcaccggcgacggcacctcctgccacctgcggttcgacccggagcgcggcacggccgtcgcgctcaccaccaacggctcgaccggcgcggcgctgtggcgggagctgtgcgccgaactggccgagcggcacgtgctgcccccggagtgggggcgcgagcccggcgaggccaccccggtcgccgggccaccggacggcgccggccactacgccaacgggcccgccgagttcgtggtgaccaggcaggacgacgacggtctcgcgctggtgctcgacggtgcccaggccgtcccgctgacctgcctgcccgacctgcgcttccagctccgcctcggcacgacggtcctgagtggacggttcctgcgcgacccggacaccggcggcatcgaccacatccaggtgaccggccgactggcccggcggcggcaggtgaccggatga

SEQ ID NO: 7 - DNA, 33 bp, Artificial Sequence
  Forward primer
ccggaattcgtgcccaacgagcaggatctcggg

SEQ ID NO: 8 - DNA, 31 bp, Artificial Sequence
  Reverse primer
cccaagctttcatccggtcacctgccgccgc

SEQ ID NO: 9 - protein, 439 aa, Streptomyces noursei
VHGDSADPAGYGAGDGSPLDLDLDRLARDCDVAGGQLAVHHHGVLETWEFGEEEPGAGRPVHSATAFPYGSTTKVCTATSVLQLVGDGDLDLDRPVREWLPEAGTVPEARRDHPALAATLRQLLSHTAGLPSDHDDADASSLRRWLTGFLAAPADARPAPGTFSYSNVGYGIAGRVVESVTGLPWYEAVRDYLLRPLGTAITALPAEPGSLPAGGLAGSAADLVRLGLLHIAEPGRPDLADPAALGEATRPTAGADPFGLADGWGPGLGHFGPAEDRWLGHDGTLDGATCHLRIHPRRGTVIALTTNSPTGQALWDAVVGALRDAGIDVGVYRPAPPPSLAQGGAPAGAAAFADCTGTYRNGDLAVTVGLDGPHLVLELPGGARELAEPLARRTFAARGAGAFLGRFAVDARTGAVHALQYSGRTLLRDTGRAHGARPH

SEQ ID NO: 10 - DNA, 1320 bp, Streptomyces noursei
gtgcacggggactcagcggatccagcggggtacggcgccggtgacggctcccccctcgacctcgacctggaccgtctcgcccgtgactgcgacgtggccggcggacagctggccgtccaccaccacggtgtgctggagacctgggagttcggcgaggaggaacccggcgccggacggccggtgcactccgcgacggccttcccctacgggtcgacgaccaaggtgtgcaccgccacctccgtcctgcaactggtcggcgacggcgacctggacctcgaccggcccgtacgggaatggctccccgaggccgggacggttcccgaagcgcggcgcgaccacccggccctcgccgccaccctccgccaactcctcagccacaccgccggactgccctccgaccacgacgacgcggacgcctcctcgctgcgccgctggctgaccggcttcctggccgcaccggccgacgcccggcccgccccgggcaccttctcctactccaacgtcggctacggcatcgccggacgcgtcgtggagtccgtcaccggactgccctggtacgaggcggtacgggactacctcctgcgcccgctgggcaccgccatcaccgcgctgcccgccgaacccggctcgctgcccgccggcgggctcgcgggcagcgccgccgacctggtgcggctgggtctgctccacatcgcggagccgggccgcccggacctggccgacccggcggccctgggggaggcgacccggcccacggccggcgccgacccgttcgggctggccgacggctggggcccgggcctgggccacttcggccccgcggaggaccgctggctcggccacgacggcacgctggacggcgccacctgtcacctgcggatccacccccggcgcggcaccgtcatcgcgctgaccaccaactccccgaccgggcaggccctgtgggacgccgtggtcggcgcactgcgggacgcgggcatcgacgtcggcgtgtaccgccccgcacccccgccgtccctcgcgcagggaggcgcccccgccggcgcggcggccttcgcggactgcacgggcacctaccgcaacggcgacctggccgtgacggtcggcctcgacggcccgcacctcgtactggaactcccgggcggggcaagggaactggccgagccgctggcccggcggaccttcgccgcccggggggccggggccttcctcgggcgcttcgccgtcgacgcgcgcaccggcgcggtccacgccctccagtacagcggccgcaccctcctgcgggacacgggccgcgcccacggcgcacggccgcactaa

SEQ ID NO: 11 - DNA, 32 bp, Artificial Sequence
  Forward primer
ccggaattcgtgcacggggactcagcggatcc

SEQ ID NO: 12 - DNA, 31 bp, Artificial Sequence
  Reverse primer
cccaagcttttagtgcggccgtgcgccgtgg

(19) United States
(12) Patent Application Publication
(10) Pub. No.: US 2021/0139917 A1
(43) Pub. Date: May 13, 2021

(54) PEPTIDE MACROCYCLIZATION ENZYME

(71) Applicant: NATIONAL UNIVERSITY CORPORATION HOKKAIDO UNIVERSITY, Hokkaido (JP)

(72) Inventors: Toshiyuki WAKIMOTO, Sapporo-shi, Hokkaido (JP); Takefumi KURANAGA, Sapporo-shi, Hokkaido (JP); Kenichi MATSUDA, Sapporo-shi, Hokkaido (JP)

(73) Assignee: NATIONAL UNIVERSITY CORPORATION HOKKAIDO UNIVERSITY, Hokkaido (JP)

(21) Appl. No.: 17/053,162

(22) PCT Filed: Apr. 25, 2019
(86) PCT No.: PCT/JP2019/017707

§ 371 Date: Nov. 5, 2020

(30) Foreign Application Priority Data
May 7, 2018 (JP) 2018-089287
Mar. 19, 2019 (JP) 2019-050797

(51) Int. Cl. C12N 15/52; C07K 14/36; C12N 15/63

(52) U.S. Cl. CPC C12N 15/52; C12N 15/63; C07K 14/36

(57) ABSTRACT

A peptide cyclase that has the amino acid sequence represented by SEQ ID NO: 1 or a mutated sequence thereof, or a peptide cyclase that has an amino acid sequence encoded by a base sequence encoding the amino acid sequence represented by SEQ ID NO: 1 or a mutated sequence thereof; DNA encoding the peptide cyclase; a method for producing the peptide cyclase; and a method for producing a cyclic peptide using the peptide cyclase.

### TECHNICAL FIELD

The present invention relates to a peptide cyclization enzyme capable of producing a macrocyclic peptide, a method for producing the enzyme, and a method for producing a cyclic peptide using the enzyme.

### BACKGROUND ART

Macrocyclic compounds can impart fixation of the steric conformation of a compound, resistance to a biodegrading enzyme, and the like, and are structural features which are commonly found particularly in physiologically active substances and medicaments derived from natural products. However, there are very limited conventional organic synthetic methods for cyclizing a peptide by efficiently connecting functional groups which are present at a ratio of 1:1 in a molecule and which are located away from each other (see Non Patent Literature 1). Finding an efficient cyclization method is one of problems which have not been solved yet in synthetic organic chemistry.

### CITATION LIST

### Non Patent Literature

Non Patent Literature 1: C. J. White, A. K. Yudin, Nature Chem. 2011, 3, 509-524

### SUMMARY OF INVENTION

### Problem to be Solved by the Invention

An problem to be solved by the present invention to find a method for cyclizing a peptide by efficiently connecting functional groups which are present at a ratio of 1:1 in a molecule and which are located away from each other.

### Means to Solve the Problem

The present inventors have extensively conducted studies for solving the above-described problem, and found that peptide cyclization enzymes derived from Actinomycetes are capable of efficiently producing a desired macrocyclic peptide, which led to completion of the present invention.

That is, the present invention provides the following (1) to (7).

(1) A peptide cyclization enzyme comprising:

(a) an amino acid sequence set forth as SEQ ID NO: 1,

(b) an amino acid sequence having a 35% or more identity to an a o acid sequence set forth as SEQ ID NO: 1, or

(c) an amino acid sequence set forth as SEQ ID NO: 1 in which one to tens of amino acid residues are deleted, substituted, inserted or added.

(2) A peptide cyclization enzyme comprising an amino acid sequence which is encoded by a DNA containing:

(a) a nucleotide sequence set forth as SEQ ID NO: 2,

(b) a nucleotide sequence which hybridizes under stringent conditions with a nucleotide sequence complementary to a nucleotide sequence set forth as SEQ ID NO: 2.

(3) A DNA comprising:

(a) a nucleotide sequence encoding an amino acid sequence set forth as SEQ ID NO: 1,

(b) a nucleotide sequence encoding an amino acid sequence having a 35% or more identity to an amino acid sequence set forth as SEQ ID NO: 1,

(c) a nucleotide sequence encoding an amino acid sequence set forth as SEQ ID NO: 1 in which one to tens of amino acid residues are deleted, substituted, inserted or added, or

(d) a nucleotide sequence which hybridizes under stringent conditions with a nucleotide sequence complementary to a nucleotide sequence encoding an amino acid sequence set forth as SEQ ID NO: 1.

(4) A DNA comprising:

(a) a nucleotide sequence set forth as SEQ ID NO: 2, or

(b) a nucleotide sequence which hybridizes under stringent conditions with a nucleotide sequence complementary to a nucleotide sequence set forth as SEQ ID NO: 2.

(5) A rector comprising the DNA described in (3) or (4).

(6) A method for producing a peptide cyclization enzyme, comprising culturing cells into which the DNA described in (3) or (4), or the vector described in (5) has been introduced, and then obtaining a peptide cyclization enzyme from the culture.

(7) A method for producing a cyclic peptide, comprising applying the peptide cyclization enzyme described in (1) or (2) to a substrate peptide.

### Effects of Invention

According to the present invention, there are provided an enzyme capable of efficiently producing a cyclic peptide, particularly a macrocyclic peptide, a method for producing the enzyme, and a method for producing a cyclic peptide using the enzyme. The peptide cyclization enzyme of the present invention has broad substrate specificity, so that it is possible to obtain cyclized peptides having various components and lengths which have been difficult to obtain by conventional organic synthetic methods. According to the present invention, various physiologically active substances and medicaments can be produced.

### BRIEF DESCRIPTION OF DRAWINGS

FIG. 1 shows the results of polyacrylamide gel electrophoresis of a peptide cyclization enzyme of the present invention (derived from Streptomyces albidoflavus NBRC 12854). Lane M represents a molecular weight marker, and lane 1 represents the peptide cyclization enzyme of the present invention.

FIG. 2a shows a reaction formula showing conversion of a substrate peptide (3) into a cyclized peptide (1) by the peptide cyclization enzyme of the present invention (derived from Streptomyces albidoflavus NBRC 12854). FIG. 2b shows a HPLC chromatogram of a reaction solution when the peptide cyclization enzyme of a the present invention is applied to the substrate peptide of FIG. 2a. Reaction mix. +1 (std.) co-injection represents a chromatogram of a sample obtained by mixing the reaction solution with a surugamide B preparation indicated with 1 in FIG. 2a. Surugamide B1 (std.) represents a chromatogram of the surugamide B preparation. Reaction mix. represents a chromatogram of the reactionsolution. Reaction mix. without enzyme represents a chromatogram of a reaction solution without addition of the peptide cyclization enzyme of the present invention. Reaction mix without substrate represents a chromatogram of a reaction solution without addition of the substrate peptide.

FIG. 3 shows a reaction formula showing a non-enzymatic cyclization reaction of the substrate peptide.

FIG. 4 shows the results of HPLC analysis of a reaction induced by the peptide cyclization enzyme of the present invention (derived from Streptomyces albidoflavus NBRC 12854) when another substrate peptide is used. In the HPLC chromatogram, + represents a chromatogram of a reaction solution with addition of the peptide cyclization enzyme of the present invention, and − represents a chromatogram of a reaction solution without addition of the peptide cyclization enzyme of the present invention. ×10 means that the boxed portion of the chromatogram is magnified by 10 times.

FIG. 5 shows the results of polyacrylamide gel electrophores s of a peptide cyclization enzyme of the present invention (derived from Goodfellowiella coeruleoviolacea NBRC 14988). Lane M represents a molecular weight marker, and lane E2 represents the peptide cyclization enzyme of the present invention.

FIG. 6 shows the results of polyacrylamide gel electrophoresis of a peptide cyclization enzyme of the present invention (derived from Streptomyces noursei NBRC 15452). Lane M represents a molecular weight marker, and lane E2 represents the peptide cyclization enzyme of the present invention.

FIG. 7 shows the results of HPLC analysis of cyclization products obtained using the peptide cyclization enzyme derived from Goodfellowiella coeruleoviolacea NBRC 14988 and the peptide cyclization enzyme derived from Streptomyces noursei NBRC 15452 when the substrate peptide 3 (SB-SNAC) shown in FIG. 2 is used as a substrate. SB-SNAC only represents a system without addition of an enzyme, +14988 represents a system with addition of the peptide cyclization enzyme derived from Goodfellowiella coeruleoviolacea NBRC 14988, and +15452 represents a system with addition of the peptide cyclization enzyme derived from Streptomyces noursei NBRC 15452.

FIG. 8 shows the results of HPLC analysis of cyclization products obtained using the peptide cyclization enzyme derived from Goodfellowiella coeruleoviolacea NBRC 14988 and the peptide cyclization enzyme derived from Streptomyces noursei NBRC 15452 when as a substrate, a peptide is used in which D-Leu at the C-terminus of the substrate peptide 3 shown in FIG. 2 is substituted with D-Ala (SB(L8A)-SNAC). SB(L8A)-SNAC only represents a system without addition of an enzyme, +14988 represents a system with addition of the peptide cyclization enzyme derived from Goodfellowiella coeruleoviolacea NBRC 14988, and +15452 represents a system with addition of the peptide cyclization enzyme derived from Streptomyces noursei NBRC 15452.

FIG. 9 shows the results of HPLC analysis of cyclization products obtained using the peptide cyclization enzyme derived from Goodfellowiella coeruleoviolacea NBRC 14988 and the peptide cyclization enzyme derived from Streptomyces noursei NBRC 15452 when as a substrate, a peptide is used in which D-Leu at the C-terminus of the substrate peptide 3 shown in FIG. 2 is substituted with D-Phe (SB(L8F)-SNAC). SB(L8F)-SNAC only represents a system without addition of an enzyme, +14988 represents a system with addition of the peptide cyclization enzyme derived from Goodfellowiella coeruleoviolacea NBRC 14988, and +15452 represents a system with addition of the peptide cyclization enzyme derived from Streptomyces ncursei NBRC 15452.

### DESCRIPTION OF EMBODIMENTS

In an aspect, the present invention provides a peptide cyclization enzyme comprising:

(a) an amino acid sequence set forth as SEQ ID NO: 1,

(b) an amino acid sequence having a 35% or more identity to an amino d sequence set forth as SEQ ID NO: 1, or

(c) an amino acid sequence formed by deletion, substitution, insertion or addition of one to tens of amino acid residues its an amino acid sequence set forth as SEQ ID NO: 1.

In the present description, the peptide cyclization enzyme is an enzyme having the action of cyclizing a peptide by binding an amino group, which is present at a portion forming the peptide (e.g. one amino acid residue), to a carboxyl group, which is present at another portion (e.g. another amino acid residue) to generate a peptide bond. The peptide cyclization enzyme of the present invention can cyclize a peptide in a head-to-tail manner, so that a large cyclic peptide can be produced.

In the present description, the amino acid is normally a natural amino acid and an L-isomer, and can encompass nonnatural amino acids such as β-alanine, D-isomer amino acids, and modified amino acids obtained by modifying amino acids by methods such as alkylation, esterification and halogenation.

Unless otherwise specified, the terms as used in the present description have meanings which are normally understood in the fields of biology, biochemistry, chemistry, pharmacy, medicine and the like.

The peptide cyclization enzyme of the present invention may contain an amino acid sequence set forth as SEQ ID NO: 1.

The peptide cyclization enzyme of the present invention may contain a variant sequence of an amino acid sequence set forth as SEQ ID NO: 1. For example, the peptide cyclization enzyme of the present invention may contain an amino acid sequence having an identity of 35% or more, for example 40% or more, preferably 50% or more, for example 60% or more, more preferably 70% or more, for example 80% or more or 85% or more, still more preferably 90% or more, for example 92% or more, 94% or more, 96% or more or 98% or more to an amino acid sequence set forth as SEQ ID NO: 1. The identity of amino acid sequences can be examined by known means such as FASTA search, BLAST search or the like.

When the peptide cyclization enzyme of the present invention contains a variant sequence of an amino acid sequence set forth as SEQ ID NO: 1, is preferable that in the variant sequence, the amino acid sequence of a portion corresponding to the 63rd to 66th amino acid residues of SEQ ID NO: 1 is Ser-X₁-X₂-Lys, and/or the amino acid sequence of a portion corresponding to the 153rd to 158th amino acid residues of SEQ ID NO: 1 is Ser-Tyr-Ser-Asn-X₃-Gly, and/or the amino acid sequence of a portion corresponding to the 304th to 307th amino acid residues of SEQ ID NO: 1 is Gly-His-X₄-Gly, and/or the amino acid sequence of a portion corresponding to the 374th to 379th amino acid residues of SEQ ID NO: 1 is Gly-X₅-X₆-X₇-Asn-Gly. More preferably, the amino acid sequence of a portion corresponding to the 374th to 379th amino acid residues of SEQ ID NO: 1 is Gly-X₅-X₆-X₇-Asn-Gly. The corresponding portion is not needed to be a portion having same amino acid numbers as the portion of SEQ ID NO: 1, and may a portion near said portion. The corresponding portion can be found by comparing the amino acid sequence with SEQ ID NO: 1. For example, a portion in the amino acid sequence of SEQ ID NO: 5, which corresponds to the 63rd to 66th amino acid residues of SEQ ID NO: 1, is Ser-Leu-Thr-Lys which corresponds to 59th to 62nd amino acid residues. X₁ to X₇ each independently represent an amino acid residue.

For example, the peptide cyclization enzyme of the present invention may contain an amino acid sequence set forth as SEQ ID NO: 1 in which one to several amino acid residues are deleted, substituted, inserted or added. However, the number of amino acid residues submitted to deletion, substitution insertion or addition in an amino acid sequence set forth as SEQ ID NO: 1 is not limited to one to several, and may be one to tens, preferably 1 to 40, more preferably 1 to 20, still more preferably one to several. The term “tens” means that the number of such amino acid residues may be, for example, 20, 30, 40, 50, 60, 70, 80 or 90. The term “several” means that the number of such amino acid residues may be, for example, 2, 3, 4, 7, 8 or 9. Deletion, substitution, insertion or addition of amino acid residues in an amino acid sequence of protein is known to those skilled in the art. Deletion, substitution, insertion or addition of amino acid residues in an amino acid sequence of the peptide cyclization enzyme of the present invention may be caused by using, for example, a site-specific mutation method or a known chemical method. In substitution of amino acid residues, substitution with homologous amino acids is preferable. The homologous amino acids are known to those skilled in the art.

The peptide cyclization enzyme the present invention which contains a variant sequence of an amino acid sequence set forth as SEQ ID NO: 1 has a cyclization activity of preferably 50% or more, more preferably 60% or more, still more preferably 70% or more, even more preferably 80% or more, most preferably 90% or more of that of the peptide cyclization enzyme of the present invention which contains an amino acid sequence set forth as SEQ ID NO: 1.

The cyclization activity can be measured by a known method. For example, the amount of a cyclic peptide produced per unit amount of enzyme and unit time may be used as an index of the cyclization activity.

In another aspect, the present invention provides a peptide cyclization enzyme comprising an amino acid sequence which is encoded by a DNA containing:

(a) a nucleotide sequence set forth as SEQ ID NO: 2 or

(b) a nucleotide sequence which hybridizes under stringent conditions with a nucleotide sequence complementary to a nucleotide sequence set forth as SEQ ID NO: 2.

The nucleotide sequence set forth as SEQ ID NO: 2 encodes an amino acid sequence set f th as SEQ ID NO: 1.

In the present description, the nucleotide sequence encompasses degenerate sequences encoding a target amino acid sequence.

The peptide cyclization enzyme of the present invention may contain an amino acid sequence which is encoded by a DNA containing a nucleotide sequence set forth as SEQ ID NO: 2.

The peptide cyclization enzyme of the present invention may contain an amino acid sequence which is encoded by a DNA containing a variant sequence of a nucleotide sequence set forth as SEQ ID NO: 2. For example, the peptide cyclization enzyme of the present invention may contain an amino acid sequence which is encoded by a DNA containing a nucleotide sequence which hybridizes under stringent conditions with a nucleotide sequence complementary to a nucleotide sequence set forth as SEQ ID NO 2.

The stringent conditions are known to those skilled in the art, and examples thereof include the following conditions:

hybridization is carried out for 16 to 24 hours under the condition of a temperature of 60 to 68° C. preferably 65° C. still more preferably 68°C. in a buffer solution containing 0.25 M Na₂HPO₄ (pH 7.2), 7% SDS, 1 mM EDTA and a 1×Denhardt's solution, and washing is performed for 1.5 minutes twice under the condition of a temperature of 60 to 68° C., preferably 65° C., still more preferably 68° C. in a buffer solution containing 20 mM Na₂HPO₄ (pH 7.2), 1% SDS and 1 mM EDTA; or

prehybridization is carried out overnight at 42° C. in a hybridization solution containing 25% formamide, or 50% formamide as a more severe condition, 4×SSC (sodium chloride/sodium citrate), 50 mM HEPES (pH 7.0) a 10×Denhardt's solution and 20 μg/ml modified salmon sperm DNA, and washing is then performed at 37° C. in a buffer solution containing 1×SSC and 0.1% SDS, at 42° C. in a buffer solution containing 0.5×SSC and 0.1% SDS as a more severe condition, or at 65° C. in a buffer solution containing 0.2×SSC and 0.1% SDS as a still more severe condition.

Of course, the stringent conditions are not limited to the above examples.

The peptide cyclization enzyme of the present invention which contains an amino acid sequence which is encoded by a DNA containing a variant sequence of a nucleotide sequence set forth as SEQ ID NO: 2 has a cyclization activity of preferably 50% or more, more preferably 60% or more, still more preferably 70% or more, even more preferably 80% or more, most preferably 90% or more of that of the peptide cyclization enzyme of the present invention which contains an amino acid sequence which is encoded by a DNA containing a nucleotide sequence set forth as SEQ ID NO: 2.

In still another aspect, the present invention provides a DNA comprising:

(a) a nucleotide sequence encoding an amino acid sequence set forth as SEQ ID NO: 1,

(b) a nucleotide sequence encoding an amino acid sequence having a 35% or more identity to an amino acid sequence set forth as SEQ ID NO: 1,

(c) a nucleotide sequence encoding an amino acid sequence set forth as SEQ ID NO: 1 in which one to tens of amino acid residues are deleted, substituted, inserted or added, or

(d) a nucleotide sequence which hybridizes under stringent conditions with a nucleotide sequence complementary to a nucleotide sequence encoding an amino acid sequence set forth as SEQ ID NO: 1.

The identity of amino acid sequences is as described above. The number of amino acid residues submitted to deletion, substitution, insertion or addition is also as described above. The stringent conditions are also as described above.

In still another aspect, the present invention provides a DNA comprising:

(a) a nucleotide sequence set forth as SEQ ID NO: 2, or

(b) a nucleotide sequence which hybridizes under stringent conditions with a nucleotide sequence complementary to a nucleotide sequence set forth as SEQ ID NO: 2.

The stringent conditions are as described above.

The DNA can be obtained by a method known to those skilled in the art. The DNA may be obtained by cloning a surf gene or a homolog or an ortholog thereof. The living organism to be used as a source of the DNA is not particularly limited, and is preferably a microorganism, more preferably Actinomycetes. For example, the DNA of the present invention may be obtained by the method described in Examples of the present description. Examples of the Actinomycetes include, but are not limited to, Streptomyces, Actinomyces, Mycobacterium, Corynebacterium and Goodfellowiella.

The DNA encodes the above-described peptide cyclization enzyme of the present invention. using the DNA, the peptide cyclization enzyme of the present invention can be produced by a genetic engineering method. For example, the peptide cyclization enzyme of the present invention may be obtained from a culture obtained by incorporating the DNA of the present invention into an expression vector, introducing the vector into cells, and culturing the cells. For example, the peptide cyclization enzyme of the present invention may be obtained from a culture obtained by introducing the DNA into cells by use of a known method such as a PEG method, electroporation or a particle gun method, and culturing the cells.

In still another aspect, the present invention provides a vector containing the DNA in the aspect described above. The vector is preferably an expression vector. Various expression vectors are known, and can be appropriately selected and used. Methods for incorporating the DNA of the present invention into a vector are also known.

In still another aspect, the present invention provides a method for producing a peptide cyclization enzyme, comprising culturing cells into which the DNA of the present invention or the vector has been introduced, and then obtaining a peptide cyclization enzyme from the culture. The cells to be used for the method in this aspect are not particularly limited, and may be cells of microorganisms such as bacteria, yeasts, filamentous bacteria or Actinomycetes, plant cells, or animal cells. Examples of preferred cells to be used for this method include, but are not limited to, cells of microorganisms such as Escherichia coil and Bacillus subtilis.

When cells are used which produce the peptide cyclization enzyme of the present invention inside cells, the cells are broken by known means such as ultrasonic waves, a mill or a homogenizer to obtain a liquid extract, from which the peptide cyclization enzyme of the present invention can be obtained by known means such as ammonia sulfate precipitation or chromatography. When cells are used which produce the peptide cyclization enzyme of the present invention outside cells, the peptide cyclization enzyme of the present invention can be obtained by subjecting a culture solution to known means such as ammonia sulfate precipitation or chromatography.

In still another aspect, the present invention provides method for producing a cyclic peptide, comprising applying the peptide cyclization enzyme of the present invention to a substrate peptide.

The peptide cyclization enzyme of the present invention has broad substrate specificity, and can cyclize even a large peptide. Therefore, the composition and the length of the substrate peptide to be used for the method of the present invention are not particularly limited. The substrate peptide may be a target cyclic peptide in which any peptide bond has been cleaved. The substrate peptide may have a length of several or more amino acids, for example 7 or more amino acids, 9 or more amino acids or 11 or more amino acids.

In the case where a cyclic peptide is obtained by using the peptide cyclization enzyme of the present invention, a cyclization reaction is accelerated when the C-terminus and/or the N-terminus of the substrate peptide are a bulky amino acid residue and/or a hydrophobic amino acid residue. Therefore, use of the peptide cyclization enzyme of the present invention enables fusion between bulky amino acid residues, which is difficult by chemical synthesis.

It is preferable from the viewpoint of improving cyclization efficiency that the carboxyl group at C-terminal of the substrate peptide used in the method of the present invention is, derivatized and activated. Examples of the derivatization include, but are not limited to, esterification. Examples of the esterification include, but are not limited to, thioesterification, and alkyl-esterification (e.g. methyl-esterification and ethyl-esterification).

By incubating a solution containing the substrate peptide and the peptide cyclization enzyme of the present invention under appropriate conditions, the peptide cyclization enzyme of the present invention can be applied to the substrate peptide. Those skilled in the art can determine the appropriate conditions according to factors such as the type and the concentration of the substrate peptide, and the amount of the enzyme used. Examples of the appropriate conditions include, but are not limited to, those of reaction carried out at room temperature to about 37° C. and a pH of 6 to 9 for several hours. The peptide cyclization enzyme may be immobilized to a carrier in use.

Production the cyclic peptide can be confirmed by known means. For example, a reaction solution may be analyzed using high performance liquid chromatography (HPLC). A product having a molecular weight of the substrate peptide cyclized may be examined using mass spectrometry. The cyclic peptide preparation may be a commercialized product, or may be obtained by chemical synthesis.

The present invention will be described below in a more detailed and specific manner by way of Examples, which should not be construed as limiting the scope of the present invention.

### EXAMPLE 1

Hereinafter, the peptide cyclization enzyme of the present invention is referred to as “SurE” or “SurE protein”.

### (1) Preparation of Plasmid for Expression of Recombinant SurE Protein

With a genome of Streptomyces albidoflavus NBRC 12854 as a template, a gene fragment encoding SurE was amplified by a PCR reaction using a primer SurE_Fw/SurE_Rv as shown below. This was treated with a restriction enzyme EcoRI/HindIII, and inserted into a multi-cloning site (MCS) of pUC19. The inserted sequence was checked, and the inserted fragment was then reinserted into an EcoRI/HindIII site of MCS of pET28 to prepare pET28-surE.

SurE_Fw:
(SEQ ID NO: 3)
CCGGAATTCCATATGGGTGCCGAGGGGCG
SurE_Rv:
(SEQ ID NO: 4)
CCCAAGCTTTCAGAGCCGGTGCATGGC

### (2) Preparation of Recombinant SurE Protein

pET28-surE was introduced into Escherichia coli (E. coli) BL21 to prepare E. coli for expression of recombinant SurE. A single colony of the E. coli was inoculated in 2xYT medium containing kanamycin (Km) at 25 μg/ml, and cultured overnight at 37° C. This was inoculated at 1.0% (v/v) in 2xYT medium containing kanamycin (Km) at 25 μg/ml, and cultured at 37° C. until OD600 was about 0.5. This was cooled on ice for 5 minutes, ITPG was then added to a final concentration of 0.1 mM, and culturing was performed overnight at 16° C. The culture solution was centrifuged at 4,000 xg, and the bacterial cells were recovered. The bacterial cells were resuspended in a buffer A (20 mM Tris-HCl (pH 8.0), 150 mM, NaCl), and centrifuged at 4,000 xg again. The bacterial cells were resuspended in the buffer A again, and ultrasonically broken. The liquid after cell breakage was centrifuged at 15,000 xg, and the resulting supernatant was subjected to Ni affinity column chromatography equilibrated with a buffer B (20 mM Tris-HCl, 150 mM NaCl, 20 mM imidazole (pH 8.0)) in advance. The resin was further washed with the buffer B, and recombinant SurE protein bound to the resin was then eluted with a buffer C (20 mM Tris-HCl (pH 8.0), 150 mM NaCl, 500 mM imidazole (pH 8.0)). The eluted protein was subjected to polyacrylamide gel electrophoresis. A single band was observed at a molecular weight of 47 kDa as shown in FIG. 1. The amino acid sequence of the resulting SurE protein is set forth as SEQ ID NO: 1, and the nucleotide sequence encoding the amino acid sequence is set forth as SEQ ID NO: 2.

### (3) Peptide Cyclization Reaction by Recombinant SurE (i)

A reaction solution having the composition of 20 mM Tris-HCl (pH 6.0), 1 mN substrate peptide and 9 μg of recombinant SurE was prepared using the recombinant SurE protein prepared by the above-described procedure and a substrate peptide (SNAC body indicated with 3 in FIG. 2a). The substrate peptide was prepared by fusing N-acetylcysteamine with a peptide synthesized by solid-phase synthesis. The reaction solution was incubated at 30° C. for 2 hours, and subjected to HPLC analysis. Here, as a column, COSMOSIL 5C18-MS-II 4.6×250 mm Column (nacalai tesque) was used, and as a mobile phase, 41% MeCN+0.05% TFC was used at a flow rate of 0.8 ml/min. The product was monitored by absorption of UV having a wavelength of 200 nm.

FIG. 2b shows the results of the HPLC analysis. The reaction solution of the recombinant SurE protein and the substrate peptide (Reaction mix. in FIG. 2b) showed a peak from a single product at a retention time of about 18.4 minutes. The retention time for this peak was identical to the retention time for the peak from a Surugamide B preparation (1 in FIG. 2) (Surugamide B1 (Std.) in FIG. 2b). A sample obtained by mixing the reaction solution with the Surugamide B preparation showed a single peak at a retention time identical to that for the peak from the Surugamide B preparation (Reaction mix. +1 (std.) co-injection in FIG. 2b). These results revealed that the peptide cyclization enzyme of the present invention (SurE) cyclized the substrate peptide to give a cyclized peptide 1 (Surugamide B) as a single product.

Without use of SurE, the substrate peptide was incubated for 2 days in the presence of Et3N (pH 12), and subjected to HPLC analysis. A peak was observed at a retention time of about 16.2 minutes, which was identical to the retention time for the compound 4 preparation of FIG. 3 (data is not shown).

These results showed that, the cyclized peptide 1 (Surugamide B) was obtained by the peptide cyclization enzyme of the present invention.

### (4) Peptide Cyclization Reaction by Recombinant SurE (ii)

SurE was applied to the substrate peptide (SNAC body) shown in FIG. 4, and the reaction solution was analyzed by HPLC and mass spectrometry (extracted ion chromatogram). Surugamide F was not detected, and a product having a molecular weight corresponding to the molecular weight (m/z 1038.8) of a cyclized product of Surugamide F (cyclic Surugamide F) was detected at a HPLC retention time of about 13.8 minutes. Even when a shorter peptide (7 amino acid residues, SNAC body) and a longer peptide (11 amino acid residues, SNAC body) as compared to the peptide used above were used, a cyclized product was observed to be obtained by the recombinant SurE of the present invention (data not shown). These results showed that the enzyme of the present invention permits a wide range of length of the substrate.

### EXAMPLE 2

### (1) Cyclization Reaction Using Substrate Having Bulky Amino Acid Residue at C-Terminus/N-Terminus

Experiments were conducted using a substrate in which D-Leu at the C-terminus of the compound indicated in 3 of FIG. 2a is substituted with bulkier D-Phe and a substrate in which IIe at the N-terminus of the compound indicated in 3 of FIG. 2a is substituted with bulkier L-Trp. Recombinant SurE obtained in Example 1 was used as an enzyme. A reaction was carried out while the concentration of the substrate was changed, the cyclized product was analyzed by HPLC, and a K_{M} value and a kcat value were calculated. The catalytic efficiency was evaluated by using a reaction rate constant kcat/K_{M}.

The kcat/K_{M} value obtained when the above-described two types of substrates were used was two to five times higher than the kcat/K_{M} value obtained when the compound indicated with 3 in FIG. 2a was used as a substrate. This result showed that the cyclization reaction was promoted when the amino acid residues at the C-terminus and/or the N-terminus of the substrate were a bulky amino acid residue and/or an amino acid residue having high hydrophobicity. It was shown that use of SurE of the present invention enables a fusion reaction between bulky amino acid residues, which is difficult in chemical synthesis.

(2) Cyclization Reaction of Recombinant SurE with Methyl Ester as Substrate

A methyl ester which is the compound indicated with 3 in FIG. 2a was obtained by extension of a peptide chain by Fmoc solid-phase synthesis, methyl-esterification using Mel, and deprotection of a Boc group. It was confirmed that even when the methyl ester was used as a substrate, the same cyclized product as in the case of using the SNAC body was produced. Since the SurE of the present invention is capable of producing a cyclized product even when a methyl ester which can be supplied more conveniently and inexpensively than the SNAC body is used as a substrate, the SurE of the present invention is considered as an economically advantageous enzyme.

The experimental results show Examples above showed that the peptide cyclization enzyme of the present invention was a useful enzyme capable of cyclizing substrate peptides having different compositions and lengths.

### EXAMPLE 3

(1) Preparation of Recombinant Peptide Cyclization Enzyme from Microorganisms Other Than Streptomyces Albidoflavus

From Goodfellowiella coeruleoviolacea NBRC 14988 and Streptomyces noursei NBRC 15452, cyclization enzymes were obtained in the same manner as in Example 1.

Primers used for cloning were as follows:

forward primer: ccggaattcgtgcccaacgagcaggatcggg (SEQ ID NO: 7) and reverse primer: cccaagatttcatccggtcacctgccgccgc (SEQ ID NO: 8) for Goodfellowiella coeruleoviolacea NBRC 14988; and

forward primer: ccggaattcgtgcacggggactcagcggatcc (SEQ ID NO: 11) and reverse primer: cccaagcttttagtgcggccgtgcgccgtgg (SEQ ID NO: 12) for Streptomyces noursei NBRC 15452.

FIGS. 5 and 6 show the results of polyacrylamide gel electrophoresis of the peptide cyclization enzymes obtained from the above-described two strains of microorganisms. The peptide cyclization enzyme from Goodfellowiella coeruleoviolacea NBRC 14988 showed a single band at 49.2 kDa, while the peptide cyclization enzyme from Streptomyces noursei NBRC 15452 showed a single band at 49.0 kDa.

The amino acid sequence of the peptide cyclization enzyme from Goodfellowiella coeruleoviolacea NBRC 14988 is set forth as SEQ ID NO: 5, and the nucleotide sequence of the peptide cyclization enzyme is set forth as SEQ ID NO: 6. The amino acid sequence of the peptide cyclization enzyme from Streptomyces noursei NBRC 15452 is set forth as SEQ ID NO: 9, and the nucleotide sequence of the peptide cyclization enzyme is set forth as SEQ ID NO: 10. The amino acid sequence of the peptide cyclization enzyme from Goodfellowiella coeruleoviolacea NBRC 14988 had a 38% identity to the amino acid sequence of SEQ ID NO: 1, and the amino acid sequence of the peptide cyclization enzyme from Streptomyces noursei NBRC 15452 had a 37% identity to the amino acid sequence of SEQ ID NO: 1.

### (2) Peptide Cyclization Reaction by Recombinant Peptide Cyclization Enzyme

The two recombinant peptide cyclization enzymes obtained as described above and substrate peptides (SNAC body: “SB-SNAC” indicated with 3 in FIG. 2, peptide “SB(L8A)-SNAC” in which D-Leu at the C-terminus of SB-SNAC is substituted with D-Ala, and peptide “SB(L8F)-SNAC” in which D-Leu at the C-terminus of SB-SNAC is substituted with D-Phe) were reacted in the same manner as in. Example 1, and the products were observed by HPLC. FIGS. 7, 8 and 9 show HPLC chromatograms using SB-SNAC as a substrate, using SB(L8A)-SNAC as a substrate, and using SB(L8F)-SNAC as a substrate, respectively.

As is apparent from FIGS. 7 to 9, the two enzymes produced cyclized peptides from three substrates. In FIG. 7, the peak at a retention time of about 11.7 minutes corresponds to a head-to-tail cyclized peptide. In FIG. 8, the peak at a retention time of about 10.3 minutes corresponds to a head-to-tail cyclized peptide. In FIG. 9, the peak at a retention time of about 11.8 minutes corresponds to a head-to-tail cyclized peptide. In FIGS. 8 and 9, peaks of two cyclized peptides were observed, and the peak at a longer retention time corresponded to an isopeptide non-enzymatically cyclized between a lysine residue and an amino acid residue at the C-terminus in the substrate peptide.

From these results, it was confirmed that a peptide cyclization enzyme was obtained even from microorganisms other than Streptomyces albidoflavus.

### INDUSTRIAL APPLICABILITY

The present invention is applicable to production of medicaments and laboratory reagents, etc.

### SEQUENCE LISTING FREE TEXT

SEQ ID NO: 1 shows an amino acid sequence of the peptide cyclization enzyme of the present invention (derived from Streptomyces albidoflavus NBRC 12854).

SEQ ID NO: 2 shows a nucleotide sequence encoding the peptide cyclization enzyme of the present invention (derived from Streptomyces albidoflavus NBRC 12854).

SEQ ID NO: 3 shows a nucleotide sequence of the forward primer used for cloning of the peptide cyclization enzyme of the present invention (derived from Streptomyces albidoflavus NBRC 12854).

SEQ ID NO: 4 shows a nucleotide sequence of the reverse primer used for cloning of the peptide cyclization enzyme of the present invention (derived from Streptomyces albidoflavus NBRC 12854).

SEQ ID NO: 5 shows an amino acid sequence of the peptide cyclization enzyme of the present invention (derived from Goodfellowiella coeruleoviolacea NBRC 14988).

SEQ ID NO: 6 shows a nucleotide sequence of the peptide cyclization enzyme of the present invention (derived from Goodfellowiella coeruleoviolacea NBRC 14988).

SEQ ID NO: 7 shows a nucleotide sequence of the forward primer used for cloning of the peptide cyclization enzyme of the present invention (derived from Goodfellowiella coeruleoviolacea NBRC 14988).

SEQ ID NO: 8 shows a nucleotide sequence of the reverse primer used for cloning of the peptide cyclization enzyme of the present invention (derived from Goodfellowiella coeruleoviolacea NBRC 14988).

SEQ ID NO: 9 shows an amino acid sequence of the peptide cyclization enzyme of the present invention (derived from Streptomyces noursei NBRC 15452).

SEQ ID NO: 10 shows a nucleotide sequence encoding the peptide cyclization enzyme of the present invention (derived from Streptomyces noursei NBRC 15452).

SEQ ID NO: 11 shows a nucleotide sequence of the forward primer used for cloning of the peptide cyclization enzyme of the present invention (derived from Streptomyces noursei NBRC 15452).

SEQ ID NO: 12 shows a nucleotide sequence of the reverse primer used for cloning of the peptide cyclization enzyme of the present invention (derived from Streptomyces noursei NBRC 15452).

1. A peptide cyclization enzyme comprising:
(a) an amino acid sequence set forth as SEQ ID NO: 1,
(b) an amino acid sequence having a 35% or more identity to an amino acid sequence set forth as SEQ ID NO: 1, or
(c) an amino acid sequence set forth as SEQ ID NO: 1 in which one to tens of amino acid residues are deleted, substituted, inserted or added.

2. A peptide cyclization enzyme comprising an amino acid sequence which is encoded by a DNA containing:
(a) a nucleotide sequence set forth as SEQ ID NO: 2, or
(b) a nucleotide sequence which hybridizes under stringent conditions with a nucleotide sequence complementary to a nucleotide sequence set forth as SEQ ID NO: 2.

3. A DNA comprising:
(a) a nucleotide sequence encoding an amino acid sequence set forth as SEQ ID NO: 1,
(b) a nucleotide sequence encoding an amino acid sequence having a 35% or more identity to an amino acid sequence set forth as SEQ ID NO: 1,
(c) a nucleotide sequence encoding an amino acid sequence set forth as SEQ ID NO: 1 in which one to tens of amino acid residues are deleted, substituted, inserted or added, or
(d) a nucleotide sequence which hybridizes under stringent conditions with a nucleotide sequence complementary to a nucleotide sequence encoding an amino acid sequence set forth as SEQ ID NO: 1.

4. A DNA comprising:
(a) a nucleotide sequence set forth as SEQ ID NO: 2, or
(b) a nucleotide sequence which hybridizes under stringent conditions with a nucleotide sequence complementary to a nucleotide sequence set forth as SEQ ID NO: 2.

5. A vector comprising the DNA according to claim 3.

6. A method for producing a peptide cyclization enzyme, comprising culturing cells into which the DNA according to claim 3, or a vector comprising the DNA, has been introduced, and then obtaining a peptide cyclization enzyme from the culture.

7. A method for producing a cyclic peptide, comprising applying the peptide cyclization enzyme according to claim 1 to a substrate peptide.

8. A vector comprising the DNA according to claim 4.

9. A method for producing a peptide cyclization enzyme, comprising culturing cells into which the DNA according to claim 4, or a vector comprising the DNA, has been introduced, and then obtaining a peptide cyclization enzyme from the culture.

10. A method for producing a cyclic peptide, comprising applying the peptide cyclization enzyme according to claim 2 to a substrate peptide.
